(19) United States
(12) Patent Application Publication
(10) Pub. No.: US 2013/0144175 A1
(43) Pub. Date: Jun. 6, 2013

(54) PERSONAL HEALTH INFORMATION IDENTIFICATION TAG

(71) Applicants: Sheldon M. Lambert, Plano, TX (US); John W. Woodmansee, JR., Frisco, TX (US)

(72) Inventors: Sheldon M. Lambert, Plano, TX (US); John W. Woodmansee, JR., Frisco, TX (US)

(21) Appl. No.: 13/309,309

(22) Filed: Dec. 1, 2011

(51) Int. Cl. A61B 5/021; A61B 5/145; G08B 1/08

(52) U.S. Cl. 600/485; 340/539.12; 600/481

(57) ABSTRACT

A smart identification tag system comprises a helmet and an identification tag. The helmet defines a headspace adapted to receive a wearer's head. A sensor assembly is disposed in the headspace and comprises a sensor capable of capturing data and a wireless transceiver in communication with the sensor and adapted to transmit a wireless signal indicative of data captured by the sensor. The identification tag comprises a wireless transceiver adapted to receive the signal from the helmet, and a non-transitory memory in communication with the wireless transceiver and adapted to store the received data. The helmet is adapted to be worn by a wearer, and the identification tag is adapted to be carried on the person of the wearer, such that information captured by the sensor assembly is transmitted to the identification tag and stored therein. The identification tag may further store personal health information of the wearer.

### CROSS-REFERENCE TO RELATED APPLICATIONS

This application claims the benefit of provisional application Ser. No. 61/418,064, Filed on Dec. 1, 2010, the entire contents of which are hereby expressly incorporated herein by reference.

### FIELD OF DISCLOSURE

The inventive concepts disclosed herein generally relate to personnel identification tags, and more particularly, but not by way of limitation, to a smart identification tag system which is worn or carried by a wearer and which stores personal heath information about the wearer and is capable of communicating with a sensor system, in order to receive and store information about forces exerted on the wearer's head and body.

### BACKGROUND

Identification tags for personnel are known in the prior art. For example, soldiers and members of similar organizations, as well as civilian employees, wear such identification tags regularly. Identification tags are essentially data carriers having data that is specific to the particular person wearing the tag (wearer).

Soldiers, in particular, wear identification tags, which are unofficially referred to as “dog tags”. At the present time, these dog tags are typically made of a metal sheet, and are hung around a soldier's neck on a chain that has a breakable weak link. A soldier's name, rank, unit, social security number, religious affiliation, and blood type, are usually engraved on the dog tag. Multiple dog tags are typically worn by a soldier, which multiple dog tags may be attached on the same chain around the soldier's neck, or worn on separate chains and/or attached to different parts of the soldier's body such as ankles, arms, wrists, neck, uniform, and boots, for example. In the event of the injury of a soldier, the dog tags remain with the soldier to provide positive identification while the soldier is being transported to various medical care facilities and treated for their injuries. On the death of a soldier, one of the dog tags is removed by breaking the weak link in the chain, and is used to report the death of the soldier in question; the other dog tags usually remain on the body for later identification.

The basis for the material selection for the manufacture of dog tags and the engraving of data is that, should the need arise, the dog tag serves to reliably provide information about a particular wearer, even under the harshest conditions, for example, after an explosion, a fire, exposure to shrapnel and/or environmental conditions, and even years in the corrosive ground.

The limited amount of data stored on such conventional dog tags can no longer meet the needs of a modern, highly technological army. In modern armed forces, a soldier is increasingly considered an independent weapons system that is also a part of a bigger weapons system. This may be evidenced by a soldier's ability, acquired from special training and equipment, to operate independently or as a member of a small team, while at the same time maintaining communications with a higher command element allowing a soldier to update mission objectives, report information, and receive support.

Information about a soldier's training, for example, parachute training, specific competencies, such as driver's license, knowledge, and a soldier's personal health information, such as prior injuries, vaccinations, and allergies, is usually available in decentralized locations in various paper and data files, and often cannot be readily retrieved in emergency situations in forward care centers, such as the military equivalent of a civilian emergency room.

Due to changes in current warfare realities, such as improvised explosive devices (“IEDs”) and the development of tactics such as using IEDs for widespread roadside bombings, a large number of soldiers sustain traumatic brain injuries in the line of duty. When a soldier has sustained an injury, or injuries, which might include traumatic brain injury, it is important to accurately diagnose the degree of the injury and provide immediate treatment to mitigate the effects of the brain injury. The first medical personnel to treat injured soldiers are usually field medics and/or field hospital staff, located in hostile zones, or nearby sanctuaries, often with limited access to the personal health information of injured soldier(s). Additionally, such first responders usually lack testing equipment used to properly diagnose brain injuries, such as Computer Assisted Tomography (CAT) scanners, Magnetic Resonance Imaging (MRI) scanners, and/or X-ray machines, for example.

To protect the head and brain of soldiers, helmets are typically worn during military activities. In recent years, smart helmet systems have been developed, in order to detect and record the severity of head injuries in military applications. Such systems typically contain a helmet-integrated sensor package that automatically collects data throughout the soldier's activities. More recent developments introduce capabilities which allow for distinguishing and recording of the exact times and severity of single or multiple events, and provide a visible and/or audible indication when a pre-set force threshold has been crossed. These smart helmet systems are usually powered by a rechargeable battery. For an example of such a smart helmet system, see the Headborne Energy Analysis and Diagnostic System (HEADS) developed by BAE Systems.

Other similar smart helmet systems have been developed for civilian applications, in order to monitor the forces experienced by a wearer's head during various activities. Such civilian smart helmets typically employ accelerometers to measure acceleration along the x, y, and z axes, and transmit such information to a remote central location. For an example of a civilian smart helmet see U.S. Pat. No. 5,978,972, the entire disclosure of which is hereby incorporated herein by reference.

In a military application, however, there exists the risk that the enemy may intercept the signal from a soldier's helmet, and use it to obtain the location and number of soldiers, as well as any other sensitive information transmitted by the helmet. There is also a need for this information to be relayed to the higher command element, which is receiving and recording this data.

Accordingly, there exists a need for a smart identification tag system that is able to provide fast access to the personal health information of a wearer, while at the same time being capable of providing information about forces exerted on the wearer's head and brain. The smart identification tag system should be capable of communicating with a smart remote sensor system located for example on, or around, the wearer's head, such as a system embedded in a fitted head band of a helmet, which is measuring the forces exerted upon the wearer's brain during mission activities, to obtain information about forces exerted on the wearer's head and/or body. To such a smart identification tag system the inventive concepts disclosed herein are directed.

### SUMMARY

In one aspect, the inventive concepts disclosed herein are directed to a smart identification tag system. The system comprises a helmet defining a headspace adapted to receive a wearer's head and a sensor assembly at least partially disposed in the headspace. The sensor assembly comprises at least one sensor capable of capturing data and a wireless transceiver in communication with the at least one sensor and adapted to transmit a wireless signal indicative of data captured by the at least one sensor. The system further comprises an identification tag having a wireless transceiver adapted to receive the wireless signal transmitted by the helmet, and a non-transitory memory in communication with the wireless transceiver and adapted to store data received by the wireless transceiver. The helmet is adapted to be worn by a wearer, and the identification tag is adapted to be carried on the person of the wearer, such that information captured by the sensor assembly is transmitted to the identification tag and stored therein.

In another aspect, the inventive concepts disclosed herein are directed to a smart dog tag, comprising a non-transitory computer medium storing information comprising personal health information of a wearer, a transceiver in communication with the non-transitory computer medium and adapted to allow information to be read from and written in the non-transitory computer medium, and a hanging assembly comprising means for attaching the non-transitory computer medium and the transceiver to the person of the wearer.

In another aspect, the inventive concepts disclosed herein are directed to a method for locating a wearer of a smart dog tag, comprising receiving a first signal via a wireless transceiver of the smart dog tag, in response to receiving the first signal, transmitting a second signal indicative of the location of the smart dog tag, and providing an alert perceivable by the wearer to remain in place for rescue.

In another aspect, the inventive concepts disclosed herein are directed to a method of treating injured persons comprising: providing a person with a smart dog tag system, which has a helmet defining a headspace, adapted to receive the person's head and a sensor assembly connected to the helmet. The sensor assembly comprises at least one sensor and a first wireless transceiver in communication with the at least one sensor and adapted to transmit a wireless signal indicative of data captured by the at least one sensor. The smart dog tag system also has a dog tag comprising a second wireless transceiver adapted to receive the wireless signal transmitted by the first wireless transceiver, and a non-transitory memory in communication with the second wireless transceiver comprising personal health information of the person and adapted to store data received by the second wireless transceiver. The helmet is worn by the person, and the dog tag is carried on the person, such that information captured by the sensor assembly is transmitted to the dog tag and stored therein. The method further comprises accessing the non-transitory memory to obtain the personal health information of the person and information from the at least one sensor and treating the person at least partially based on the information obtained from the dog tag.

In yet another aspect, the inventive concepts disclosed herein are directed to a smart helmet system comprising a helmet defining a headspace adapted to receive a wearer's head and a sensor assembly disposed on the helmet. The sensor assembly comprises at least one sensor and a wireless transceiver in communication with the at least one sensor and adapted to transmit a wireless signal indicative of data captured by the at least one sensor. The wireless signal is provided with a signal strength sufficient to limit reception to a distance of about four feet or less.

### BRIEF DESCRIPTION OF THE DRAWINGS

Like reference numerals in the figures represent and refer to the same element or function. Implementations of the disclosure may be better understood when consideration is given to the following detailed description thereof. Such description makes reference to the annexed pictorial illustrations, schematics, graphs, drawings, and appendices. In the drawings:

FIG. 1 is a perspective view of a smart identification tag system according to the present disclosure.

FIG. 2 is a perspective view of a helmet system according to the present disclosure.

FIG. 3 is a block diagram of a helmet sensor assembly according to the present disclosure.

FIG. 4 is a diagram of a dog tag system according to the present disclosure.

FIG. 5 is a top plan view of a dog tag assembly manufactured in accordance with the present disclosure.

FIG. 6 is a side view of the dog tag assembly shown in FIG. 5 in its closed state.

FIG. 7 is a side view of the dog tag assembly shown in FIG. 5 in its open state.

### DETAILED DESCRIPTION

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. The inventive concepts disclosed herein are capable of other embodiments or of being practiced or carried out in various ways. Also, it is to be understood that the phraseology and terminology employed herein is for the purpose of description and should not be regarded as limiting the inventive concepts in any way.

In the following detailed description of embodiments of the disclosure, numerous specific details are set forth in order to provide a more thorough understanding of the inventive concepts disclosed herein. However, it will be apparent to one of ordinary skill in the art that the inventive concepts within the instant disclosure may be practiced without these specific details. In other instances, well-known features have not been described in detail to avoid unnecessarily complicating the instant disclosure.

The inventive concepts disclosed herein are directed to a smart identification tag system containing personal health information about a wearer and adapted to be worn on, or about, the person of the wearer. The smart identification tag system may hereinafter be interchangeably referred to as “Personal Health Information Dog Tag” or “PHI dog tag system.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

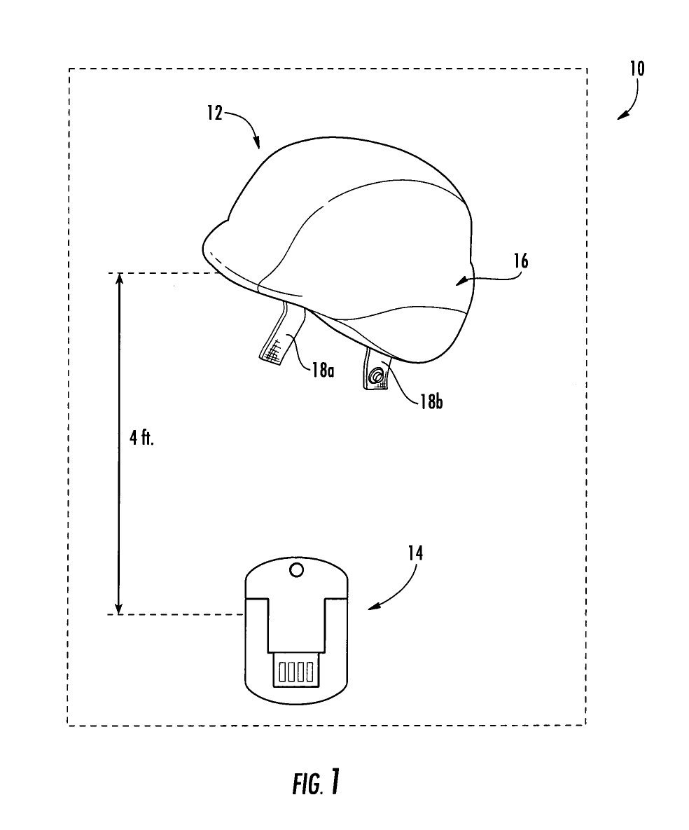

Referring now to FIG. 1, shown therein is a PHI dog tag system 10 according to the inventive concepts disclosed herein. Generally, the PHI dog tag system 10 comprises a helmet system 12 and a dog tag system 14. The helmet system 12 is adapted to measure forces experienced by a wearer and transmit a wireless signal over a short distance. The wireless signal comprises information about the forces measured by the helmet system 12. The distance over which the wireless signal is transmitted is desirably about four feet or shorter, in order to avoid signal detection and/or interception by opposing forces. The dog tag system 14 is adapted to receive a\ wireless signal transmitted by the helmet system 12, and is capable of storing at least part of the information carried by the signal.

The helmet system 12 comprises a helmet assembly 16 and straps 18a and 18b.

The straps 18a and 18b are securely attached to the helmet assembly 16, and are releaseably securable to one another to allow for the fastening of the helmet assembly 16 on a wearer's head. The straps 18a and 18b can be securely fastened under a wearer's chin, neck, and face, for example. The straps 18a and 18b may have an optional chin guard (not shown), and may be fastened by any means known in the art, such as buckles, hook and loop fasteners, strings, and combinations thereof, for example. The straps 18a and 18b can be made from any suitable material such as leather, nylon, polyester, rubber, cotton, and material of the type sold as Kevlar®, for example. The straps 18a and 18b may have adjustable lengths. It is to be understood that in some embodiments, more than two straps may be used to secure the helmet assembly 16 to a wearer's head.

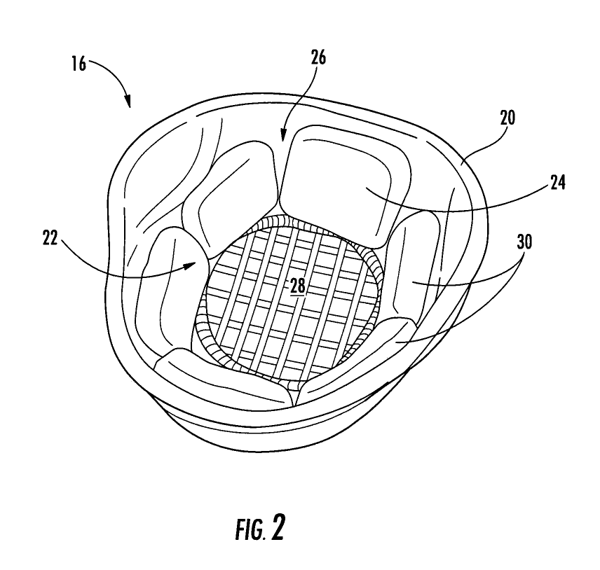

Referring now to FIG. 2, the helmet assembly 16 comprises a helmet 20, a head support assembly 22, and a sensor assembly 24.

The helmet 20 can be any helmet known in the prior art. The helmet 20 can be made of any suitable material, such as steel, other metals, material of the type sold as Plexiglas®, carbon fiber, epoxy resins, thermoplastics, and material of the type sold as Kevlar®, for example. The helmet 20 desirably has a headspace 26 formed therein, the headspace 26 adapted to allow a wearer to removably insert their head inside the headspace 26, in order to wear the helmet 20. The helmet 20 may have an optional face guard (not shown), or means (not shown) for attaching various accessories such as night vision goggles, heads-up displays, hearing protectors, or protective eyewear, for example.

The head support assembly 22 is securely attached to the helmet 20 and is at least partially disposed inside the headspace 26. The head support assembly 22 comprises a head cushioning lattice 28, and head support pads 30. The head-cushioning lattice 28 is made from any suitable material having the desired durability and elasticity. The head support pads 30 are filled with elastomeric material as is known in the art, in order to cushion the head of the wearer. The head-cushioning lattice 28 and the head support pads 30 cooperate to retain the wearer's head inside the headspace 26, while at the same time absorbing and dissipating vibrations and/or forces applied to the exterior of the helmet 20. The head support assembly 22 may be adjustable for a custom fit around a particular wearer's head, and may have moisture-wicking and/or antibacterial properties, for example.

The sensor assembly 24 is desirably integrated with the head support assembly 22, such that it does not occupy space in the headspace 26 additional to the space occupied by the head support assembly 22. It is to be understood however that the sensor assembly 24 may be unattached to the head support assembly 22 and may be housed inside a tight-fitting cap (not shown) or band (not shown), which is to be placed on the wearer's head prior to inserting the head into the helmet 20. The sensor assembly 24 may also be housed inside the headspace 26 separate from the head support assembly 22, for example.

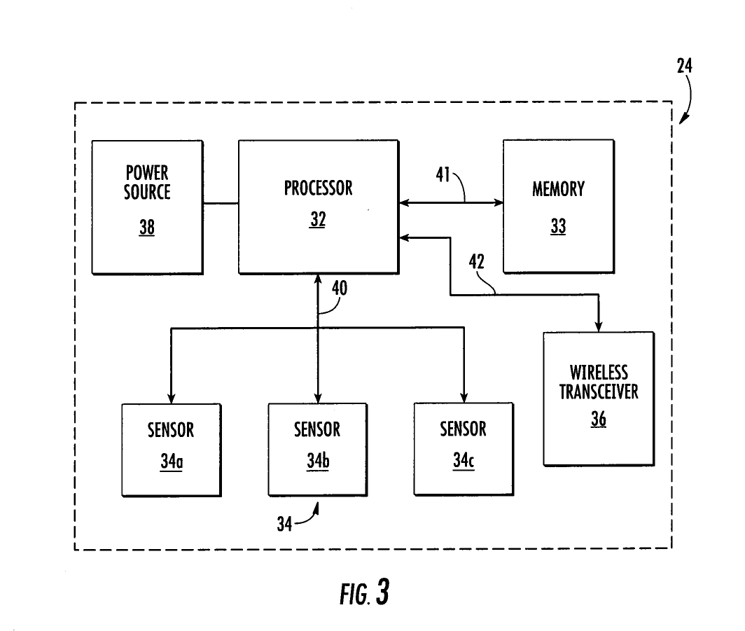

Referring now to FIG. 3, the sensor assembly 24 comprises a processor 32, a memory 33, sensors 34, a wireless transceiver 36, and a power source 38.

The processor 32 can be any processor known in the art. For example, the processor 32 can be implemented as one or more microprocessors, one or more microcontrollers, one or more field-programmable gate arrays, and/or one or more digital signal processors. It is to be understood that the processor 32 may comprise one, two, three, or a plurality of: microprocessors, microcontrollers, field-programmable gate arrays, digital signal processors, for example, or any combinations thereof. The processor 32 is capable of communicating with the sensors 34 via signal path 40. The processor 32 is capable of communicating with the memory 33 via signal path 41. The processor 32 can record forces measured by the sensors 34 into memory 33. The processor 32 is further able to read information from memory 33, and is able to execute instructions contained therein. The processor 32 communicates with the wireless transceiver 36 via a signal path 42.

The sensor assembly 24 also has a wireless transceiver 36 for wirelessly transmitting information over a relatively short distance, desirably about four feet or less, to the dog tag system 14. The wireless transceiver 36 can be any transceiver known in the art. For example, the wireless transceiver may use Wi-Fi® or Bluetooth® technology. The wireless transceiver 36 may comprise a wireless transmitter. The wireless transceiver 36 desirably transmits an unsecured signal over a short distance, in order to lower the power consumption of the helmet system 12. Alternatively, the wireless transceiver 36 may transmit a secured signal over the same or a longer distance, such as an encrypted signal, for example.

The memory 33 can be any read/write non-transitory memory known in the art, such as static or dynamic Random Access Memory (RAM), or flash memory, for example. The memory 33 may have varying capacity. The memory 33 may also comprise processor executable instructions to be executed by the processor 32. It is to be understood that while memory 33 is shown as integrated into the sensor assembly 24, the use of a removable memory carrier, such as a flash card, or a SD-card, for example, is also contemplated with the inventive concepts disclosed herein. Additionally, memory 33 may optionally be accessible through the exterior of the helmet 20.

The sensor assembly 24 can comprise at least one sensor 34 capable of measuring forces applied to the wearer's head in at least one direction. The sensor assembly 24 desirably comprises three accelerometers 34a, 34b, and 34c, arranged along three perpendicular axes x, y, and z, in order to measure forces along multiple directions. The three accelerometers 34a, 34b, and 34c are disposed so that data descriptive of the translational, angular, and normal accelerations experienced by the wearer's head are sensed. In other embodiments, any number of accelerometers measuring forces applied in any number of directions can be used. An example of a nine-accelerometer system is disclosed in U.S. Pat. No. 5,978,972, the entire disclosure of which is hereby incorporated herein by reference.

It is to be further understood that the sensor assembly 24 may comprise any number of accelerometers, and may optionally comprise other sensors such as temperature sensors, blood oxygen level sensors, noise level sensors, radiation sensors, and GPS position sensors, for example.

The power source 38 supplies power for the sensor assembly 24 and the wireless transceiver 36. The power source 38 can be one or more of a rechargeable battery, a cell phone battery, or a conventional battery, for example. The power source 38 desirably provides enough power for the sensor assembly 24 to operate for twenty-four, or more, hours on a single charge, in order to accommodate extended duty hours. To that end, an unsecured signal over a short distance is desirable, in order to minimize power consumption and maximize the operating time of the sensor assembly 24 between charges. The power source 38 can be recharged, or replaced, during rest time or after completion of the mission. Additionally, back-up power sources can be available to soldiers during long missions.

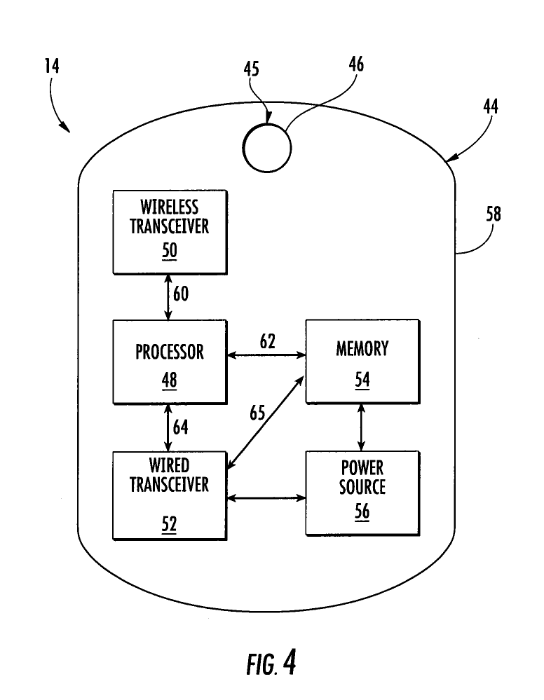

Referring now to FIG. 4, the dog tag system 14 desirably has a dog tag assembly 44 and a hanging assembly 45 for attaching the dog tag assembly 44 to the wearer's person.

The dog tag assembly 44 desirably comprises a processor 48, a wireless transceiver 50, a wired transceiver 52, a read/write memory 54, a power source 56, and a protective housing 58.

The processor 48 can be any processor known in the art. For example, the processor 48 can be implemented as one or more microprocessors, one or more microcontrollers, one or more field-programmable gate arrays, and/or one or more digital signal processors. It is to be understood that the processor 48 may comprise one, two, three, or a plurality of: microprocessors, microcontrollers, field-programmable gate arrays, digital signal processors, for example, or any combinations thereof. The processor 48 is adapted to write and read information from the read/write memory 54 via signal path 62, and is capable of executing instructions stored in memory 54. The processor is further able to communicate with the wired transceiver 52 via signal path 64.

The wireless transceiver 50 can be implemented as any conventional transceiver, and may use Wi-Fi® or Bluetooth® technology, for example. The wireless transceiver 50 is desirably adapted to communicate with the processor 48 via signal path 60. The dog tag assembly 44 is desirably adapted to receive a transmission signal from the helmet system 12, the transmission signal desirably comprising information about at least the forces exerted upon the helmet system 12. The information may be transmitted in real time, or not, and may be processed by the processor 32 helmet system 12 prior to transmission, or be unprocessed. Additionally, the wireless transceiver 50 may be connectable to a network modem, to allow for connecting the dog tag system 14 to a secured or unsecured network, and for transmitting data from memory 54 or receiving data into memory 54 over a network, such as a LAN, a wireless network, a VPN, an extranet, a WAN, a 3G network, a 4G network, or the Internet, and combinations thereof, for example.

The wired transceiver 52 is shown as a USB Standard Type A plug as a non-limiting example. It is to be understood, however, that the wired transceiver can be any wired transceiver known in the art, such as a USB plug, a USB Standard Type B plug, a USB Micro-AB Socket OTG, IBM Ultra Port, USB Mini port, IEEE 1284 port, eSATAp port, eSATA port, Ethernet port, Firewire port, HDMI port, or USB micro port, for example. It is to be further understood that the wired transceiver 52 may be attached directly to an external device (not shown), or a cable may be used to connect an external device (not shown) to the wired transceiver 52. In some embodiments, the wired transceiver 52 may be omitted, and data stored in the dog tag system 14 may be accessed via the wireless transceiver 50, for example.

An external device (not shown) can be connected to the wired transceiver 52, may be able to communicate with the processor 48 via the wired transceiver 52, and may also be able to directly access the memory 54 via signal path 65. The external device can be implemented as a conventional computer, a laptop computer, a handheld reader, a charging station, a cell phone, a Personal Digital Assistant, and a tablet, for example. Additionally, the external device may be a network modem, to allow for connecting the dog tag system 14 to a secured or unsecured network, and for transmitting data from memory 54 or receiving data into memory 54 over a network, such as a LAN, a wireless network, a VPN, an extranet, a WAN, a 3G network, a 4G network, or the Internet, and combination thereof, for example.

The memory 54 can be any conventional non-transitory read/write memory 54 capable of storing computer executable code, such as static or dynamic Random Access Memory (RAM), or flash memory, for example. The memory 54 can store information received from the sensor assembly 24 through the wireless transceiver 50. The memory 54 can further store a wearer's personal health information, including but not limited to medical history, allergies, previous injuries, previous surgeries, and blood type, for example. The memory 54 desirably has enough capacity to store the personal health information of the wearer, and twenty-four or more hours of the data received from the sensor assembly 24. The memory 54 can be compartmentalized to provide different locations for different packets of data. For example, the personal health information of the wearer may be kept in a separate location in the memory 54 from the sensor assembly 24 data. As yet another example, the sensor assembly 24 data may be divided into several locations organized by date, or in any other desired manner. The data stored on the memory 54 can be unsecured, in order to lower the power consumption of the dog tag system 14, but it is to be understood that a security protocol, such as encoding, scrambling, and encrypting the data, for example, can be used. While the memory 54 is shown as integrated into the protective housing 58 of the dog tag assembly 44, it is to be understood that the use of a removable memory carrier, such as a flash card, or a SD-card, for example, is contemplated to be used with the inventive concept(s) disclosed herein. It is to be further understood that the memory 54 may comprise processor-executable instructions to be executed by the processor 48.

The power source 56 can be one or more of a rechargeable battery, a cell phone battery, or a conventional battery, and combinations thereof, for example. The power source 56 desirably provides enough power for the dog tag assembly 44 to operate for twenty-four, or more hours, on a single charge, in order to accommodate extended duty hours. The power source may be recharged through the wired transceiver 52, or may be removed and replaced with a fresh power source 56. Alternatively, a charging station, or a charger may be provided to recharge the power source 56, or a fresh power source may be used to replace the power source 56.

The protective housing 58 serves to protect the components of the dog tag assembly 44 from moisture, sweat, vibration, impact, abrasion, and shock, for example. The protective housing 58 can be made from any suitable material, such as thermoplastic, stainless steel, polymers, and epoxy resins for example, provided that at least a portion of the protective housing 58 allows a wireless signal from wireless transceiver 36 to be received by wireless transceiver 50 and/or a signal from the wireless transceiver 50 to be transmitted to the wireless transceiver 36.

The hanging assembly 45 desirably comprises a loop and a chain (not shown). The loop is shown as an aperture 46 formed into the protective housing 58 of the dog tag assembly 44 allowing for the insertion of a chain (not shown), but it is to be understood that a loop (not shown), or any other suitable means may be used. The chain (not shown) can have a weak link built-in, in order to allow the chain to break when a pre-determined amount of force is applied thereto. It is to be understood that other means for hanging the dog tag assembly 44 can be used, such as string, rope, thread, adhesives, stitching, laces, pockets, and pouches, for example.

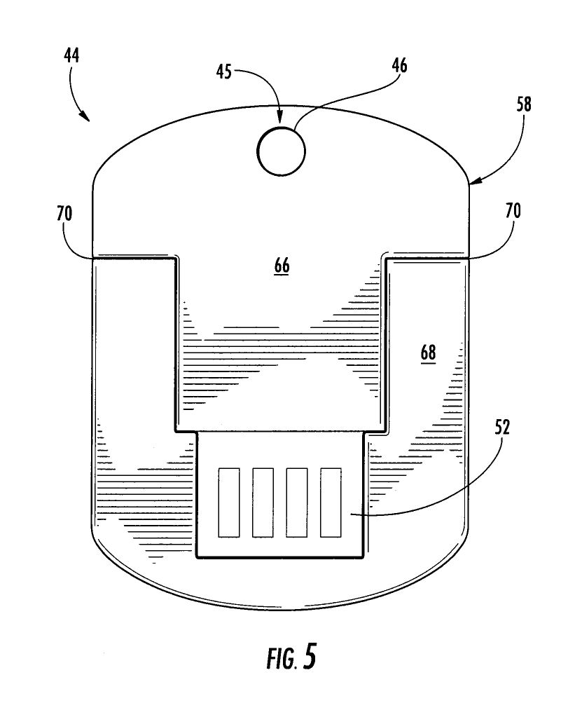

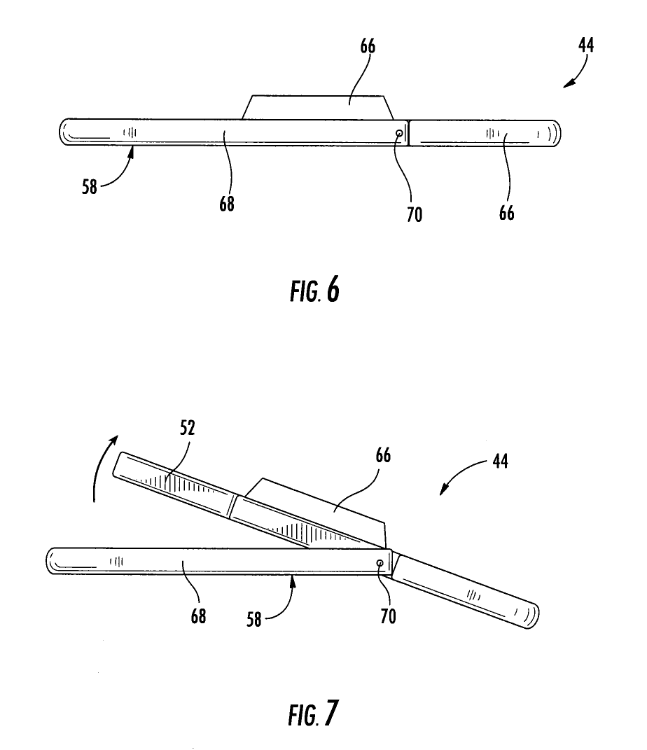

Referring now to FIGS. 5-7, shown therein is an embodiment of the protective housing 58 of the dog tag assembly 44 according to the inventive concepts disclosed herein, having a first portion 66, and a second portion 68. The first portion 66 is rotatably joined to the second portion 68 via a pin 70. This arrangement allows the protective housing 58 to be placed in a closed position (see FIG. 6), wherein the first portion 66 and the second portion 68 fit snugly with one another and substantially seal the protective housing 58. When the protective housing 58 is placed in its closed position, the first portion 66 and the second portion 68 desirably releaseably lock together, such that a predetermined amount of force has to be applied to separate the first portion 66 from the second portion 68. The protective housing 58 can also be placed in an open position (see FIG. 7), wherein the first portion 66 may be rotated relative to the second portion 68 about the pin 70. When the protective housing 58 is in its open position, the wired transceiver 52 can be exposed, so that the wired transceiver 52 can be accessed by an external device (not shown). The protective housing 58 protects the remaining components of the dog tag assembly 44 from moisture while the protective housing 58 is in its open state. It is to be understood that other configurations of the protective housing 58 can be used with the inventive concept(s) disclosed herein, such as a snap-on cap, a screw-on cap, a pouch, a pocket, or a rubber flap, for example. It is to be further understood that the protective housing 58 may comprise more than two portions, or may comprise a single portion.

It is to be understood that while a single dog tag assembly 44 is discussed herein in detail, a PHI dog tag system 10 according to the inventive concepts disclosed herein can comprise two, three, or more dog tag assemblies 44. It is to be further understood that in order to avoid interference between two or more PHI dog tag systems 10 of two or more distinct wearers, each PHI dog tag system 10 can use a unique frequency and/or unique signal identifying characteristics, in order to ensure that the signal transmitted from a helmet system 12 is uniquely identified by the dog tag system 14 that is part of the same PHI dog tag system 10 as the transmitting helmet system 12. For example, passive RFID tags, active RFID tags, and battery assisted passive (BAP) RFID tags can be used to match the helmet system 12 to a corresponding dog tag system 14.

It is to also be understood that the helmet system 12 may transmit data to the dog tag system 14 continuously, or intermittingly, for example. For example, the helmet system 12 and/or the dog tag system 14 may enter into a “sleep” mode when no forces of pre-determined magnitude are measured and/or transmitted for a certain period of time, in order to conserve power. Upon sensing a significant force, the helmet system 12 may wake up, send a “wake up” signal to the dog tag system 14, and begin transmitting data in real time, for example.

It is to further be understood that while the dog tag system 14 is shown as having its own power source 56, the dog tag system 14 may have no independent power source, but may rely on an external power source, such as inductive loop coupling from the helmet system 12. A combination of an independent power source, active RFID, passive RFID, and/or battery assisted passive (BAP) RFID technologies is also contemplated for use with the present disclosure.

In an exemplary embodiment of the instant inventive concepts, a dog tag system 14 may further comprise one or more sensors (not shown) adapted to sense and output data from various events, such as gravitational forces, concussive forces, or other forces experienced by the dog tag system 14, the geographical location of the dog tag system 14 (e.g., determined via a GPS chip), the blood pressure, heart rate, breathing rate, blood oxygen levels, blood sugar levels, and other data about the wearer, for example. Similarly, in one embodiment, the PHI dog tag system 10 may be configured to automatically provide an injured wearer alert, when certain pre-determined thresholds have been exceeded with respect to concussive forces, or other signs of injury are detected, such as low blood pressure, low heart rate, and combinations thereof, for example.

As a non-limiting example, a wearer can use the PHI dog tag system 10 by wearing the helmet system 12 on their head, and attaching the dog tag system 14 to their person and/or equipment. The distance between the helmet system 12 and the dog tag system 14 may be kept to about four feet or less, in order to minimize power consumption of the helmet system 12. During the mission activities, the helmet system 12 can sense forces experienced by the helmet 20. The sensed data can optionally be recorded in a memory 33 inside the helmet system 12. Data is also wirelessly transmitted to the dog tag system 14, where the data is stored. If a wearer is injured, or upon completion of the mission, the dog tag system 14 can be retrieved, the wired transceiver 52 can be exposed from the protective housing 58 as described above, and inserted into a portable reader or a conventional computer. The memory 54 can be accessed and the wearer's personal health information and/or the data from the helmet system 12 can be downloaded and analyzed. The downloaded data can be deleted from the memory 54, or retained therein. The power source 56 may be recharged or replaced as needed.

In a non-limiting embodiment, if a wearer is lost or injured, the dog tag system 14 may respond to a search and rescue signal, uniquely encoded for that particular PHI dog tag system 10, by transmitting its location, coordinates, or providing a beacon signal, and combinations thereof, for example, to ensure that the injured or lost wearer is rescued by appropriate personnel.

Further, in some embodiments, upon receiving a unique search signal designed, encoded, or otherwise reserved, for a specific dog tag system 14 of a missing wearer (and no other), the dog tag system 14 would recognize the search signal, and respond by emitting a signal including its location (e.g. latitude and longitude, grid coordinates), or a beacon signal allowing rescuers to determine a vector to the missing wearer, and an estimated distance based on the strength of the signal transmitted by the dog tag system 14, for example. Further, the dog tag system 14 may proactively initiate the transmission of a rescue signal unique to the particular dog tag system 14, in response to sensing a certain level of concussive force to the wearer's head or body, a drop in blood pressure, a drop in blood oxygen level, a lack of movement for a predetermined amount of time, and combinations thereof, for example.

In an exemplary embodiment, a PHI dog tag system 10 may continuously monitor the vital signs of a wearer, including but not limited to, pulse rate, breathing rate, blood pressure, body temperature, brain temperature, and blood oxygen level, and may emit an alert or emergency signal to alert the wearer, or other local or remote personnel, when one or more of the monitored vital signs is approaching or has deviated from a preset safe range. A specific reading of the particular vital sign, which is deviating from the safe range, may likewise be transmitted to a centralized location, for example. If the amount by which the threshold is deviating from the safe range is significant, a lost or injured wearer protocol may be initiated by the dog tag system 14, as described above, and a local audible, visible, and/or tactile alert may be provided to the wearer to remain in place and await rescue personnel, for example.

The inventive concepts disclosed herein have numerous civilian applications as will be appreciated by persons of ordinary skill in the art, including but not limited to car racing, motorcycle racing, boat racing, aeronautics, space flights, contact sports, winter sports, disaster recovery operations, mining operations, construction, and other activities.

It is to be understood that while a helmet system 12, and a dog tag system 14 are described in detail above, one or more of: a headband, a hat, a ski mask, a shirt, a jacket, a vest, pants, boots, shoes, gloves, a hazmat suit, a biohazard suit, a flight suit, a space suit, a wristwatch, a pocket watch, a ring, a bracelet, a necklace, a cell-phone, a personal digital assistant, a mp3 player, a tablet, a laptop, a backpack, and a radiation suit may be used in a PHI dog tag system 10 according to the inventive concepts disclosed herein. Forces sensed may be applied to different parts of the body of the wearer and maybe sensed at different locations on the wearer's body, for example.

The data collected may include radiation exposure levels, temperature, blood oxygen levels, altitude, geographical position, blood pressure, heart rate, blood glucose level, and speed, for example.

An exemplary embodiment of the PHI dog tag system 10 may optionally comprise providing an external indication when a pre-determined threshold of forces has been sensed, or in response to a pre-set triggering event, such as a visible light, a noticeable vibration, a color change indicator, and/or an audible signal. One or more external indication may be provided by the helmet system 12, by the dog tag system 14, or both, for example.

As it will be appreciated by persons of ordinary skill in the art, changes can be made in the construction and the operation of the various components, elements and assemblies described herein, or in the steps or the sequence of steps of the methods described herein, without departing from the scope of the inventive concepts disclosed herein.

From the above description, it is clear that the inventive concepts disclosed herein are well adapted to carry out the objects and to attain the advantages mentioned above, as well as those inherent in the inventive concepts disclosed herein. While presently preferred embodiments of the inventive concepts disclosed herein have been described for purposes of this disclosure, it will be understood that numerous changes may be made which will readily suggest themselves to those skilled in the art and which are accomplished within the scope of the inventive concepts disclosed and claimed herein.

What is claimed is:

1. A smart identification tag system comprising:
a helmet defining a headspace adapted to receive a wearer's head;
a sensor assembly at least partially disposed in the headspace, the sensor assembly comprising at least one sensor capable of capturing data and a wireless transceiver in communication with the at least one sensor and adapted to transmit a wireless signal indicative of data captured by the at least one sensor;
an identification tag comprising a wireless transceiver adapted to receive the wireless signal transmitted by the helmet, and a non-transitory memory in communication with the wireless transceiver and adapted to store data received by the wireless transceiver; and
wherein the helmet is adapted to be worn by a wearer, and the identification tag is adapted to be carried on a person of the wearer, such that information captured by the sensor assembly is transmitted to the identification tag and stored therein.

2. The smart identification tag system of claim 1, wherein the identification tag further comprises a wired transceiver and wherein data stored on the identification tag may be accessed by an external device via the wired transceiver.

3. The smart identification tag system of claim 1, wherein the sensor assembly comprises at least one accelerometer adapted to measure forces applied to the wearer's head.

4. The smart identification tag system of claim 1, wherein the non-transitory memory of the identification tag further comprises personal health information of the wearer.

5. The smart identification tag system of claim 1, wherein the sensor assembly further comprises a power source adapted to power the sensor assembly.

6. The smart identification tag system of claim 1, wherein the helmet further comprises a face guard.

7. The smart identification tag system of claim 1, wherein the identification tag further comprises a GPS sensor adapted to detect a position of the identification tag.

8. The smart identification tag system of claim 1, wherein the sensor assembly further comprises a blood pressure sensor.

9. The smart identification tag system of claim 1, wherein the sensor assembly further comprises a blood oxygen level sensor.

10. The smart identification tag system of claim 1, wherein the sensor assembly further comprises a temperature sensor.

11. A smart dog tag, comprising:
a non-transitory computer medium storing information comprising personal health information of a wearer;
a transceiver in communication with the non-transitory computer medium and adapted to allow information to be read from and written in the non-transitory computer medium; and
a hanging assembly comprising means for attaching the non-transitory computer medium and the transceiver to the person of the wearer.

12. The smart dog tag of claim 11 wherein personal health information is stored in the non-transitory computer medium in a format accessible by an external device via the transceiver.

13. The smart dog tag of claim 11, further comprising a wireless transceiver adapted to receive data from a sensor assembly worn by the wearer and store the received data in the non-transitory computer medium.

14. The smart dog tag of claim 11, further comprising a GPS chip adapted to detect a location of the smart dog tag, and record the location in the non-transitory computer medium.

15. A method for locating a wearer of a smart dog tag, comprising:
receiving a first signal via a wireless transceiver of the smart dog tag;
in response to receiving the first signal, transmitting a second signal indicative of the location of the smart dog tag; and
providing an alert perceivable by the wearer to remain in place for rescue.

16. The method of claim 15, wherein the first signal uniquely identifies the smart dog tag.

17. The method of claim 15, further comprising in response to receiving the first signal transmitting a third signal, comprising a beacon signal indicative of the location of the smart dog tag.

18. A method of treating injured persons comprising:
providing a person with a smart dog tag system comprising:
a helmet defining a headspace adapted to receive a person's head;
a sensor assembly connected to the helmet, the sensor assembly comprising at least one sensor and a first wireless transceiver in communication with the at least one sensor and adapted to transmit a wireless signal indicative of data captured by the at least one sensor;
a dog tag, comprising a second wireless transceiver adapted to receive the wireless signal transmitted by the first wireless transceiver, and a non-transitory memory in communication with the second wireless transceiver comprising personal health information of the person and adapted to store data received by the second wireless transceiver;
wherein the helmet is worn by the person, and the dog tag is carried on the person, such that information captured by the sensor assembly is transmitted to the dog tag and stored therein;
accessing the non-transitory memory to obtain the personal health information of the person and information from the at least one sensor; and
treating the person at least partially based on the information obtained from the dog tag.

19. The method of claim 18, wherein the at least one sensor comprises an accelerometer, and wherein information from the at least one sensor comprises information indicative of forces experienced by the person's head.

20. A smart helmet system comprising:
a helmet defining a headspace adapted to receive a wearer's head;
a sensor assembly disposed on the helmet, the sensor assembly comprising at least one sensor and a wireless transceiver in communication with the at least one sensor and adapted to transmit a wireless signal indicative of data captured by the at least one sensor; and
wherein the wireless signal is provided with a signal strength sufficient to limit reception to a distance of about 4 feet or less.

21. The smart helmet system of claim 20, wherein the helmet further comprises a processor and a non-transitory memory storing processor executable code, which when executed by the processor cause the processor to record data from the at least one sensor in the non-transitory memory.

22. The smart helmet system of claim 21, wherein the non-transitory memory is accessible by an external device.
